(19) United States
(12) Patent Application Publication
(10) Pub. No.: US 2011/0038940 A1
(43) Pub. Date: Feb. 17, 2011

(54) PULVERULENT COMPOSITION AND A PROCESS FOR PREPARING THE SAME

(75) Inventor: Alain Grizeau, La Rochelle (FR)

Correspondence Address: YOUNG & THOMPSON, 209 Madison Street, Suite 500, Alexandria, VA, 22314, US

(73) Assignee: INNOV'IA, La Rochelle (FR)

(21) Appl. No.: 12/865,927

(22) PCT Filed: Feb. 6, 2009
(86) PCT No.: PCT/EP09/51384

§ 371 Date: Nov. 2, 2010

(30) Foreign Application Priority Data
Feb. 6, 2008 (EP) 08290105.9

(51) Int. Cl. A61K 9/50; A61K 9/00; A61K 31/355; A61K 31/525; A61K 31/59; A61K 31/07; A61K 31/4188; A61K 31/519; A61K 31/714; A23L 1/302

(52) U.S. Cl. 424/493; 424/490; 514/458; 514/251; 514/167; 514/725; 514/387; 514/252.16; 514/52; 426/72

(57) ABSTRACT

A pulverulent composition includes less than 96% in weight, preferably from 10 to 80%, and more preferably from 20% to 70%, of at least a vitamin having a solubility lower than 20 g/L and preferably 1 g/L and more preferably lower than 500 mg/L in aqueous media, the pulverulent composition being stable on storage and having instantaneous dispersion and solubility properties in aqueous solutions.

The present invention relates to a pulverulent composition and to a process for preparing the same.

More particularly, the invention relates to stable pulverulent compositions having instantaneous dispersion and solubility properties in aqueous solutions, said pulverulent compositions containing substances, in particular vitamins, which are poorly soluble in aqueous media.

Some vitamins, such as vitamin E, vitamin D (D2 Ergocalciferol, D3 cholecalciferol), vitamin A and pro vitamin A, vitamin B8 (Biotin), vitamin B9 (Folic Acid), vitamin B12 (cyanocobalamin and other cobalamin) and vitamin B2 (Riboflavin) (French Patent FR 1 211 662), do not disperse well in aqueous solution. When those vitamins are added to food products or drinks to prepare vitamin supplemented food product or drinks, some lumps or spots are observed on the surface of the food product or on the wall of the container containing the drink, which is not pleasant for the consumer.

One of the aspects of the present invention is to provide a pulverulent composition having instantaneous dispersion and solubility properties in aqueous solutions.

One of the aspects of the present invention is to provide a process of preparation of a pulverulent composition having instantaneous dispersion and solubility properties in aqueous solutions.

One of the aspects of the present invention is to provide foodstuff and drinks containing a pulverulent composition having instantaneous dispersion and solubility properties in aqueous solutions.

The present invention relates to a pulverulent composition comprising less than 96% in weight, preferably from 10 to 80%, and more preferably from 20 to 70%, in particular from 30 to 60%, particularly 44 to 55%, of at least a vitamin having a solubility lower than 20 g/L, particularly lower than 10 g/L, in particular lower than 5 g/L, and preferably lower than 1 g/L and more preferably lower than 500 mg/L in aqueous media, said pulverulent composition being stable on storage and having instantaneous dispersion and solubility properties in aqueous solutions.

The present invention relates to a pulverulent composition comprising more than 15% in weight, and less than 96% in weight, preferably from 15 to 80%, and more preferably from 20 to 70%, in particular from 30 to 60%, particularly 44 to 55%, of at least a vitamin having a solubility lower than 20 g/L, particularly lower than 10 g/L, in particular lower than 5 g/L, and preferably lower than 1 g/L and more preferably lower than 500 mg/L in aqueous media, said pulverulent composition consisting in particles individually coated by at least one hydrophilic agent, and said pulverulent composition being stable on storage and having instantaneous dispersion and solubility properties in aqueous solutions.

By “pulverulent composition” is meant a composition of one or several substances in powder form.

By “vitamin” is meant a nutrient that is an organic compound required in tiny amounts for essential metabolic reactions in a living organism.

By “aqueous medium” is meant a medium in which the major component is water, in particular an aqueous solution.

By “stable on storage” is meant that the pulverulent composition is stable when stored in the original unopened packaging in a dry room (with a hygroscopicity lower than 65% and a temperature comprised between 10 and 25° C.) for one year.

By “instantaneous dispersion in aqueous solutions” is meant that when the pulverulent composition is poured into the aqueous solutions, it disperses entirely and instantaneously. For instance, for 10 g of powder added to 100 ml distilled water at room temperature, the dispersibility is characterised by the time needed for the powder to be wet by the water and disperse into the water, not standing at the surface of the water. If the time needed for wettability by water and dispersion is shorter than 15 seconds the dispersibility is considered as instant.

By “instantaneous solubility properties in aqueous solutions” is meant that the pulverulent composition dissolves entirely in the aqueous medium to make a solution. For instance, for 10 g of powder added to 100 ml distilled water at room temperature, the solubility is characterised by the quantity of lumps or insoluble particles in the water 10 nm after adding the powder in the water. No lumps at the surface and no insoluble particles at the bottom of the recipient.

The invention relates to a pulverulent composition as described above, wherein the vitamin is chosen among the group consisting of vitamin E, vitamin B2 (Riboflavin), vitamin D (D2 Ergocalciferol, D3 cholecalciferol), vitamin A and pro vitamin A, vitamin B8 (Biotin), vitamin B9 (Folic Acid), and vitamin B12 (cyanocobalamin and other cobalamin), preferably wherein the vitamin is riboflavin.

Said vitamins present a low solubility in aqueous medium.

Riboflavin has a solubility of 70 mg/L water.

The invention relates to a pulverulent composition as described above, wherein the vitamin is chosen among the group consisting of vitamin E, vitamin B2 (Riboflavin), vitamin A and pro vitamin A, vitamin B8 (Biotin), vitamin B9 (Folic Acid), and vitamin B12 (cyanocobalamin and other cobalamin), preferably wherein the vitamin is riboflavin

The invention relates to a pulverulent composition as described above, comprising at least one saccharide, chosen among the group consisting of monosaccharide, disaccharide and polysaccharide.

The invention relates to a pulverulent composition as described above, wherein the hydrophilic agent comprises or is constituted by at least one saccharide, in particular chosen among the group consisting of monosaccharides, disaccharides and polysaccharides.

By “saccharide” is meant sugar, also called carbohydrates. Monosaccharides consist of one sugar. Examples of monosaccharides include glucose (dextrose), fructose, galactose, xylose and ribose. Monosaccharides are the building blocks of:
disaccharides (consisting of two sugar) like sucrose (common sugar) and,
polysaccharides (such as cellulose and starch). Polysaccharides are polymers made up of many monosaccharides joined together by glycosidic bonds.

The present invention relates to a pulverulent composition as described above, wherein the hydrophilic agent comprises or consists in:

at least a first saccharide possessing hydrocolloidal properties,

said saccharide being preferably chosen among the group consisting of cellulose, alginate, starch, modified starch, in particular octenyl succinate starch, arabic gum, and,

at least a second saccharide, chosen among the group consisting of
monosaccharide such as fructose, glucose, galactose,
disaccharide such as saccharose, sucrose, lactose, maltose, trehalose and cellobiose, and,
polysaccharide such as glucose syrup, dextrin, inulin, cellulose, glycogen and starch or modified starch, fructooligosaccharide (FOS).
The expression “hydrocolloidal properties” means forming colloidal dispersion, and/or forming colloidal gel when dispersed in water.
The first saccharide makes a matrix around the substance or vitamin thanks to its hydrocolloidal properties, and the second saccharide participates to the matrix of the first saccharide to complete the good properties of the pulverulent composition with respect to its behaviour in water.

The present invention relates to a pulverulent composition as described above, wherein the pulverulent composition comprises from 5% to 95%, preferably 20% to 70%, in particular 30 to 60%, and more preferably from 40% to 60% in weight of saccharide.

The present invention relates to a pulverulent composition as described above, wherein the pulverulent composition comprises from 5% to 85%, preferably 20% to 70%, in particular 30 to 60%, and more preferably from 40% to 60% in weight of saccharide.

The present invention relates to a pulverulent composition as described above, wherein the vitamin is riboflavin, the first saccharide is octenyl succinate starch and the second saccharide is glucose syrup.

By “starch” is meant a mixture of amylose and amylopectin (chemical formula (C₆H₁₀O₅)ₙ). These are both complex carbohydrate polymers of glucose.

By “octenyl succinate starch” is meant octenyl succinate starch with a degree of substitution comprised between 0.01 and 0.10, preferably between 0.01 and 0.04, and more preferably around 0.02.

Octenyl succinate starch is used for its specific emulsifying properties allowing good dispersion of the matrix around each particle of substance or vitamin. Glucose sirup with its smaller glucose polymer chains complete the matrix between the spaces left by the longer chains of starch and participate to the water absorption when the pulverulent composition is rehydrated.

The present invention relates to a pulverulent composition as described above, wherein the mean diameter of said particle is from 0.2 μm to 50 μm, preferably 0.5 μm to 30 μm, more preferably 1 μm to 20 μm.

The present invention relates to a pulverulent composition as described above, characterized in that the pulverulent composition comprises granules, said granules consisting in a set of particles, and wherein:

said granules comprise at least a vitamin having a solubility lower than 20 g/L and preferably 1 g/L and more preferably lower than 500 mg/L in aqueous media and at least a saccharide,

the mean diameter of said granule is from 10 μm to 500 μm, preferably 20 to 100 μm, more preferably 30 to 50 μm,

the span (DV05) of said granules is from 0.2 to 3.0, preferably 1.0 to 2.0, more preferably from 1.2 to 1.7,

the macroscopic density of said pulverulent composition is from 300 to 800 g/l, preferably 400 to 700 g/l, and more preferably 400 to 600 g/l,

the flow index determined by Flowdex method of said pulverulent composition is from 4 to 20, preferably from 5 to 10, more preferably around 8.

It is relevant to make a distinction between the terms particles and granules.

A particle is a solid constituted by one single substance. The particle shape is defined by its origin, its chemical composition and its manufacturing process (e.g. a crystal obtained by crystallization, a dried drop obtained by a drying process, individual fiber obtained through grinding).

The granule is the macroscopic form of the pulverulent composition.

The granules are constituted by a set of particles. Said particles may be identical or different from each other.

Span is a dispersion parameter obtained in granulometer measurement by laser diffraction using three median parameters of the particle distribution curve criteria (90% Passing diameter D(v,0.90), 50% passing diameter D(v,0.50) and 10% passing diameter D(v,0.10)) as follows:

Span=[D(v,0.10)−D(v,0.90)]/D(v,0.50).

The “Flowdex method” measures the flowability of a powder measuring the smallest hole diameter of a stainless steel disc where the powder can flow, which is the Flowdex index. Flowdex device is composed of a cylinder with the interchangeable discs with holes of various diameters at the bottom. The determination of fluidity is based on the capacity of the powder to fall freely by a hole in the disc. The powder is carefully charged on the top of the hole. If the hole through which a powder falls freely is small, the flowability is good, when the hole is larger, the worse is the flowability.

The method FLOWDEX initially developed and discussed by Gioia A. Intrinsic flowability: a new technology for powder flowability classification, Pharmaceut. Technol. (1980) 1-4

The pulverulent composition as described above is the one obtained after the atomisation process as detailed hereafter.

The present invention relates to a pulverulent composition as described above, wherein said granules comprise riboflavin, modified starch, in particular octenyl succinate starch and glucose syrup.

The present invention relates to a pulverulent composition as described above, characterized in that the pulverulent composition comprises granules, wherein:

said granules comprise at least a vitamin having a solubility lower than 20 g/L and preferably 1 g/L and more preferably lower than 500 mg/L in aqueous media and at least a saccharide,

the mean diameter of said granule is from 10 μm to 500 μm, preferably 20 to 100 μm, more preferably 30 to 50 μm,

the span (DV05) of said granules is from 0.2 to 3.0, preferably 1.0 to 2.0, more preferably from 1.2 to 1.7,

the macroscopic density of said pulverulent composition is from 300 to 800 g/l, preferably 400 to 700 g/l, and more preferably 400 to 600 g/l,

the flow index determined by Flowdex method of said pulverulent composition is from 4 to 20, preferably from 5 to 10, more preferably around 8, the stability of a solution made of 1.5 g of said pulverulent composition in 8.5 g of water is such that a sediment from 1.5 ml to 10 ml, preferably 4 to 9 ml, more preferably from 6 to 8 ml is obtained after 24 hours.

By “sediment” is meant any particulate matter that can be transported by fluid flow and which eventually is deposited as a layer of solid particles on the bed or bottom of a body of the solution.

By “obtained after 24 hours” is meant 24 hours after the pulverulent composition was poured into water.

The pulverulent composition as described above is the one obtained after the microgrinding and atomisation process as detailed hereafter.

The present invention relates to a pulverulent composition as described above, wherein said granules comprise riboflavin, modified starch, in particular octenyl succinate starch and glucose.

The pulverulent composition is composed of granules, which are the unit element of the pulverulent composition.

The present invention also relates to a granule comprising at least a vitamin having a solubility lower than 20 g/L and preferably 1 g/L and more preferably lower than 500 mg/L in aqueous media, and at least a saccharide wherein:

the mean diameter of said granule is from 10 μm to 500 μm, preferably 20 to 100 μm, more preferably 30 to 50 μm,

the macroscopic density of said granule is from 300 to 800 g/l, preferably 400 to 700 g/l, and more preferably 400 to 600 g/l,

the time to disperse and solubilize 10 grams of said granules in 100 mL of an aqueous solutions is from 2 min to 5 s, preferably 1 min to 5 s, and more preferably less than 1 min, said granule comprising preferably riboflavin, glucose and modified starch, in particular octenyl succinate starch.

In another embodiment, the present invention also relates to a process of preparation of pulverulent compositions as described above and of granules constituting said pulverulent compositions as described above comprising:

1) possibly a step of mixing a vitamin having a solubility lower than 20 g/L and preferably 1 g/L and more preferably lower than 500 mg/L in aqueous media with at least a saccharide to obtain an aqueous phase comprising a vitamin and at least one saccharide, and,
2) possibly a step of microgrinding of the aqueous phase comprising a vitamin and at least one saccharide to obtain a microgrinded aqueous phase comprising a vitamin and at least one saccharide, and,
3) a step of atomisation of the microgrinded aqueous phase comprising a vitamin and at least one saccharide to obtain a mixture comprising a vitamin and at least one saccharide, said mixture being constituted by granules;
the (possibly microgrinded) aqueous phase comprising a vitamin and at least one saccharide is poured into an atomisation tower and is transformed into a mixture; said mixture is a solid and appears like a dust, constituted of granules; said mixture is either the final product, i.e. the pulverulent composition according to the invention, or an intermediary when a step of coating of the granules is added to the process (i.e. step 4));
4) possibly a step of coating of said granules to obtain a pulverulent composition, and,
5) possibly a step of recovery of the pulverulent composition obtained.

In another embodiment, the present invention also relates to a process of preparation of pulverulent compositions and of granules constituting said pulverulent compositions as described above comprising a step of atomisation of an aqueous phase comprising a vitamin and at least one saccharide, said vitamin having a solubility lower than 20 g/L and preferably 1 g/L and more preferably lower than 500 mg/L in aqueous media.

By “Atomisation”, also called “spray drying” is meant conversion of the aqueous phase comprising a vitamin and of at least one saccharide into a spray or mist (i.e. collection of drops), by passing the aqueous phase through a nozzle.

When appropriate ingredients are used in the aqueous phase, in particular saccharide, this process allows the preparation of a pulverulent composition having instantaneous dispersion and solubility properties in aqueous solutions.

The present invention also relates to a process of preparation of pulverulent compositions as described above and of granules constituting said pulverulent compositions as described above comprising:

a step of mixing a vitamin having a solubility lower than 20 g/L and preferably 1 g/L and more preferably lower than 500 mg/L in an aqueous media with at least a saccharide to obtain an aqueous phase comprising a vitamin and at least one saccharide, and,

a step of atomisation of said aqueous phase comprising a vitamin and at least one saccharide to obtain a mixture comprising a vitamin and at least one saccharide, said mixture being constituted by granules, and,

possibly a step of coating of said granules to obtain a pulverulent composition.

By “coating” is meant a covering that is applied to the granule to protect it or change its appearance. Such coating can be hydrocolloids as cellulose ingredients allowing protection against off flavors, or gums allowing gastro-resistant properties of the pulverulent composition as examples.

The present invention also relates to a process as described above in which a step of microgrinding of the aqueous phase comprising a vitamin and at least one saccharide is added prior to the step of atomisation.

By “microgrinding” is meant reducing the size of the particle to micronic size (less than 50 μm and preferably less than 10 μm).

The present invention also relates to a process of preparation of pulverulent compositions as described above and of granules constituting said pulverulent compositions as described above comprising:

a step of mixing a vitamin having a solubility lower than 20 g/L and preferably 1 g/L and more preferably lower than 500 mg/L in aqueous media with at least a saccharide to obtain an aqueous phase comprising a vitamin and at least one saccharide, and,

possibly a step of microgrinding of the aqueous phase comprising a vitamin and at least one saccharide to obtain a microgrinded aqueous phase comprising a vitamin and at least one saccharide, and,

a step of atomisation of the microgrinded aqueous phase comprising a vitamin and at least one saccharide to obtain a mixture comprising a vitamin and at least one saccharide, said mixture being constituted by granules, and,

possibly a step of coating of said granules to obtain a pulverulent composition, and,

a step of recovery of the pulverulent composition obtained.

In this embodiment, the pulverulent composition is recovered at the end of the process.

In another embodiment, the present invention relates to the use of a pulverulent composition as described above or/and of granules constituting said pulverulent compositions as described above to prepare:

liquid food products such as infantile milk, milk, acidified milk products, drinks, fruit juices and sorbet, or,

solid food products such as bakery, noodles, bread, or,

semi-liquid food products such as spreads, yoghourts, dips and ice creams, or,

emulsion food products such as sauces, in particular mayonnaise and mustard,—

powders such as dehydrated soup, dehydrated sauce, dehydrated drink preparation, powder milk, cocoa powder, instant powder drinks, meal replacement products or effervescent products, said powders being liable to be instantaneously dispersed in an aqueous solution to provide a beverage,

a pharmaceutical composition.

The advantage of using the pulverulent composition or/and the granules according to the invention in the preparation of liquid food products, solid food products, semi-liquid food products, emulsion food products, powders, and of a pharmaceutical composition is that the appearance of these products/powders/composition will be improved in comparison with products/powders/composition for the preparation of which a vitamin having a solubility lower than 20 g/L and preferably 1 g/L and more preferably lower than 500 mg/L in aqueous media is used. The colour will be more homogeneous. Fewer or no lighter or darker spots or dots will be observed on the surface or inside the products/powders/composition, which is more pleasant for the consumer.

The pharmaceutical composition contains excipient and an active substance, which is either the vitamin having a solubility lower than 20 g/L and preferably 1 g/L and more preferably lower than 500 mg/L in aqueous media, or another component of the pulverulent composition according to the invention.

In another embodiment, the present invention relates to a liquid food product containing:

from 0.1 mg/kg to 1500 mg/kg, preferably 1 mg/kg to 400 mg/kg, more preferably 4 mg/kg to 40 mg/kg of a pulverulent composition as described above,

from 0.1 mg/kg to 1500 mg/kg, preferably 1 mg/kg to 400 mg/kg, more preferably 4 mg/kg to 40 mg/kg of granules constituting said pulverulent compositions as described above, wherein:
said liquid food product is liquid or frozen,
said liquid food product is stable at temperatures from 6 to 60° C., preferably from 10° C. to 50° C., more preferably from 20° C. to 40° C. during a period of 2 years,
said pulverulent composition or granule is homogeneously dispersed in said liquid food product,
said vitamin does not form any deposit on the wall of a container which would contain said liquid food product,
said liquid food product being preferably chosen among the group consisting of oil,

infantile milk, milk, acidified milk products, drinks, fruit juices and sorbet, except liquid food product chosen from the group consisting of infantile milk, milk, acidified milk products, drinks and oils and containing:
either a pulverulent composition,
or granules constituting said pulverulent composition,
   said pulverulent composition or said granules comprising:

riboflavin,

a first saccharide possessing hydrocolloidal properties, and/or

a second saccharide, chosen among the group consisting of
monosaccharide such as fructose, glucose, galactose,
disaccharide such as saccharose, sucrose, lactose, maltose, trehalose and cellobiose, and,
polysaccharide such as glucose syrup, dextrin, inulin, cellulose, glycogen, starch and modified starch, fructooligosaccharide (FOS).

In another embodiment, the present invention also relates to a solid food product containing:

from 1 mg/kg to 1000 g/kg, preferably 10 mg/kg to 500 g/kg, more preferably 0.1 g/kg to 100 g/kg of a pulverulent composition as described above, or,

from 1 mg/kg to 1000 g/kg, preferably 10 mg/kg to 500 g/kg, more preferably 0.1 g/kg to 100 g/kg of granules constituting said pulverulent compositions as described above, wherein:
said solid food product is stable at temperatures from 6 to 60° C., preferably from 10° C. to 50° C., more preferably from 20° C. to 40° C. during a period of 2 years,
said pulverulent composition or granule is homogeneously dispersed in said solid food product,
said solid food product being preferably chosen among the group consisting of confectionery, bakery, noodles, bread, rice, cereals,
   except solid food product containing:
either a pulverulent composition,
or granules constituting said pulverulent composition,
   said pulverulent composition or said granules comprising:
riboflavin,
a first saccharide possessing hydrocolloidal properties, and/or
a second saccharide, chosen among the group consisting of
   monosaccharide such as fructose, glucose, galactose,
   disaccharide such as saccharose, sucrose, lactose, maltose, trehalose and cellobiose, and,
   polysaccharide such as glucose syrup, dextrin, inulin, cellulose, glycogen, starch and modified starch, fructooligosacchride (FOS).

In another embodiment, the present invention also relates to a semi-liquid food product containing:

from 0.1 mg/kg to 1500 mg/kg, preferably 1 mg/kg to 400 mg/kg, more preferably 4 mg/kg to 40 mg/kg of a pulverulent composition as described above,

from 0.1 mg/kg to 1500 mg/kg, preferably 1 mg/kg to 400 mg/kg, more preferably 4 mg/kg to 40 mg/kg of granules constituting said pulverulent compositions as described above, wherein:
said semi-liquid food product is semi-liquid or frozen,
said semi-liquid food product is stable at temperatures from 6 to 60° C., preferably from 10° C. to 50° C., more preferably from 20° C. to 40° C. during a period of 2 years,
said pulverulent composition or granule is homogeneously dispersed in said semi-liquid food product,
said semi-liquid food product being preferably chosen among the group consisting of spreads, jams, yoghourts, dips and ice creams,
   except semi-liquid food product chosen from the group consisting of spreads and jams and containing
either a pulverulent composition,
or granules constituting said pulverulent composition,
   said pulverulent composition or said granules comprising:
riboflavin,
a first saccharide possessing hydrocolloidal properties, and/or
a second saccharide, chosen among the group consisting of
   monosaccharide such as fructose, glucose, galactose,
   disaccharide such as saccharose, sucrose, lactose, maltose, trehalose and cellobiose, and,
   polysaccharide such as glucose syrup, dextrin, inulin, cellulose, glycogen, starch and modified starch, fructooligosaccharide (FOS).

In another embodiment, the present invention also relates to an emulsion food product containing:

from 0.1 mg/kg to 1500 mg/kg, preferably 1 mg/kg to 400 mg/kg, more preferably 4 mg/kg to 40 mg/kg of a pulverulent composition as described above,

from 0.1 mg/kg to 1500 mg/kg, preferably 1 mg/kg to 400 mg/kg, more preferably 4 mg/kg to 40 mg/kg of granules constituting said pulverulent compositions as described above, wherein:
said emulsion food product is liquid or frozen,
said emulsion food product is stable at temperatures from 6 to 60° C., preferably from 10° C. to 50° C., more preferably from 20° C. to 40° C. during a period of 2 years,
said pulverulent composition or granule is homogeneously dispersed in said emulsion food product,
said emulsion food product is a sauce, in particular mayonnaise and mustard.

In another embodiment, the present invention also relates to a powder containing:
from 1 mg/kg to 999 g/kg, preferably 10 mg/kg to 500 g/kg, more preferably 0.1 g/kg to 100 g/kg of a pulverulent composition as described above, or,
from 1 mg/kg to 999 g/kg, preferably 10 mg/kg to 500 mg/kg, more preferably 0.1 g/kg to 100 g/kg of granules constituting said pulverulent compositions as described above, and,

at least a foodstuff being preferably chosen among the group consisting of dehydrated soup, dehydrated sauce, dehydrated drink preparation, powder milk, cocoa powder, instant powder drinks, meal replacement products and effervescent products, wherein:
said powder is stable at temperatures from 6 to 60° C., preferably from 10° C. to 50° C., more preferably from 20° C. to 40° C. during a period of 2 years,
said pulverulent composition or granule is homogeneously dispersed in said powder,
said powder is liable to be instantaneously dispersed in an aqueous solution to provide a beverage,
   except powders containing:
either a pulverulent composition or granules constituting said pulverulent composition, said pulverulent composition or said granules comprising:
   riboflavin,
   a first saccharide possessing hydrocolloidal properties, and/or
   a second saccharide, chosen among the group consisting of
      monosaccharide such as fructose, glucose, galactose,
      disaccharide such as saccharose, sucrose, lactose, maltose, trehalose and cellobiose, and,
      polysaccharide such as glucose syrup, dextrin, inulin, cellulose, glycogen, starch and modified starch, fructooligosaccharide (FOS), and,
a foodstuff being chosen among the group consisting of powder milk, cocoa powder, instant powder drinks, meal replacement products and effervescent products.

The present invention also relates to a powder as described above, wherein said foodstuff is soluble in an aqueous solution and preferably chosen among the group consisting of dehydrated drink preparation, instant powder drinks and effervescent products, and wherein said powder is liable to be dissolved in an aqueous solution to provide a beverage.

In this embodiment, as the foodstuff and the pulverulent composition and/or the granules are soluble in the aqueous solution, the beverage reconstituted by mixing the powder with an aqueous solution is a homogeneous solution just after said mixing is done. No suspension, spot, dot or emulsion is observed in the beverage.

In another embodiment, the present invention also relates to a process of preparation of a liquid food product as described above, comprising a step of instantaneously dispersing a powder as described above in an aqueous solution.

In this embodiment, the beverage which is obtained by mixing the powder according to the invention with an aqueous solution, is the liquid food product according to the invention.

The present invention also relates to a process as described above, comprising an additional phase of dissolving a powder as described above in an aqueous solution.

In this embodiment, the beverage, which is obtained by mixing the powder according to the invention with an aqueous solution and is a homogeneous solution, is the liquid food product according to the invention.

In another embodiment, the present invention also relates to a pharmaceutical composition containing:

an active substance contained in the pulverulent composition as described above, or,

an active substance contained in granules as described above,

in association with a pharmaceutically acceptable vehicule.

The pulverulent composition according to the invention contains the active principle of the pharmaceutical composition. In an embodiment, said active principle is the vitamin having a solubility lower than 20 g/L and preferably 1 g/L and more preferably lower than 500 mg/L in aqueous media. In another embodiment, said active principle is another compound comprised in the pulverulent composition according to the invention, and the vitamin is one of the excipient.

In another embodiment, the present invention also relates to a pharmaceutical composition as described above, wherein the pulverulent composition or the granules comprise(s) riboflavin.

In an embodiment, the active principle is riboflavin. In another embodiment, the active principle is another compound comprised in the pulverulent composition according to the invention, and riboflavin is one of the excipient.

### FIGURES

FIG. 1:

FIG. 1 represents pure riboflavin observed on Electronic Scanning Microscopy.

FIG. 2:

FIG. 2 represents the pulverulent composition A according to the invention observed on Electronic Scanning Microscopy.

FIG. 3:

FIG. 3 represents the sedimentation measured by turbidity using a back scattering laser device (Turbiscan MA 1000) of a dispersion of the pulverulent composition A according to the invention compared to the one of pure riboflavin. The percentage of back scattering is represented as a function of time.

### EXAMPLES

### Example 1

### Ingredients of the Pulverulent Compositions A and B

The riboflavin water-dispersible pulverulent compositions A and B have been made in accordance with the invention and contain the ingredients disclosed in Table 1.

TABLE 1
Ingredients of the pulverulent compositions A and B.

| Ingredient | % by total weight | Raw material reference |
|---|---|---|
| Riboflavin | 43.00 | Riboflavin (Hubei Guangji |
| | | Pharmaceutical) |
| OSA | 14.25 | Sodium Octenyl Succinate starch |
| | | (Cerestar) |
| Glucose syrup | 42.75 | Glucose syrup DE 38 (Cerestar) |

% is based on dry matter of each component

Pulverulent composition A was obtained after spray drying and pulverulent composition B was obtained after microgrinding followed by spray drying.

### Example 2

### Detailed Protocol for Manufacture of Water-Dispersible Pulverulent Composition A Obtained by Spray Drying

The ingredients of the composition are the ones disclosed in example 1.

0.76 kg of sodium octenyl succinate (OSA) and 2.26 kg of glucose syrup are added to 4.82 kg of water and complete dissolution is achieved. 2.16 kg of riboflavin are added and stirred vigorously to obtain a uniform suspension. The above suspension is then spray dried at a rate of 11 kg per hour. The inlet air (air coming in the atomisation tower) temperature is held at about 150° C. and the outlet (air coming out of the atomisation tower) temperature is about 100° C. The pulverulent composition A obtained disperses instantaneously in water and results in a stable suspension before uses.

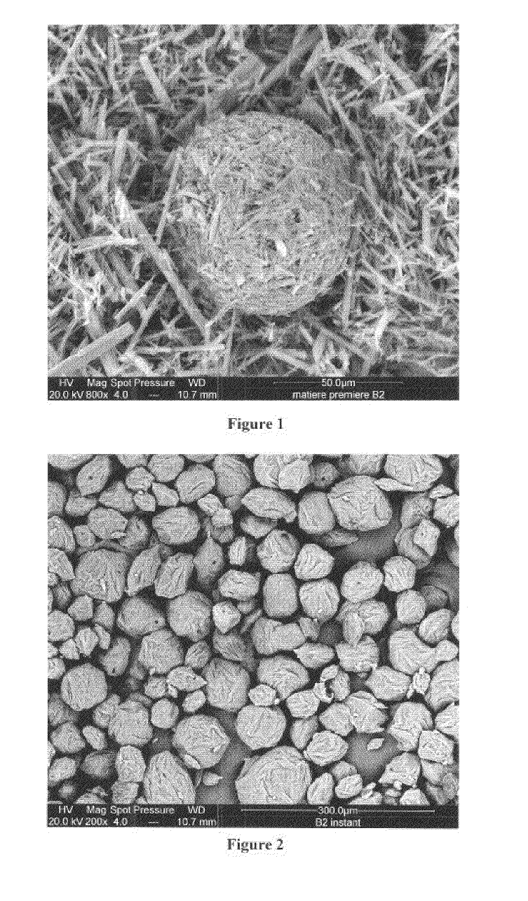

Granules of the pulverulent composition A obtained with the invention have been observed on Electronic Scanning Microscopy and the encapsulation can be observed surrounding the insoluble vitamin. The granules according to the invention obtained have a shape which is different from the one of pure vitamin (FIGS. 1 and 2).

### Example 3

### Protocol for Testing the Water-Dispersible Pulverulent Composition a of Example 2

The stability of the solution of the pulverulent composition A obtained in example 2 in water can be determined by the following laboratory tests.

A—Sedimentation of Dispersed Pulverulent Composition A in Water Measured by Turbidity.

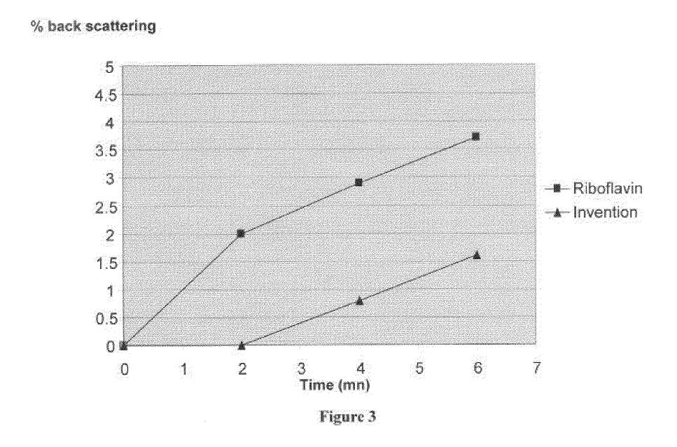

10 g of the water-dispersible pulverulent composition A is dispersed in 90 ml of distilled water at 20° C. in a tube. The sedimentation rate is measured by turbidity using a back scattering laser device (Turbiscan MA 1000) along the tube. The back light is measured along the tube at different time so that evolution of the sedimentation can be followed along time. The sedimentation is measured at the bottom of the tube. The following results show that the pulverulent composition A sediments at a lower rate compared to the control, which is the sedimentation of pure riboflavin (Table 2 and FIG. 3).

TABLE 2
% Back light after 2 and 6 minutes according
to the nature of the composition.

| | % Back light after | % Back |
|---|---|---|
| | 2 min | light after 6 min |
| composition | dispersion | dispersion |
| 100% Riboflavin | 2% | 3.1% |
| Water-dispersible | 0% | 1.6% |
| pulverulent composition A of | | |
| example 2 | | |

B—Sedimentation of Dispersed Pulverulent Composition A in Water Measured by Natural Sedimentation in Tube.

The water-dispersible pulverulent composition A is dispersed in distilled water at 20° C. at a concentration of 15% (net w/total w) in a conical graduated sedimentation tube (total volume of the solution is 10 ml). The quantity of the sediment (in ml) is measured along time (Table 3).

The encapsulation efficiency can also be assessed by measuring the hydration of the granules of the water-dispersible pulverulent composition of the invention. This has been evaluated by measuring the dry solid content of the sediment (% dry matter) (Table 3).

TABLE 3
Sediment volume and percentage of dry solids content
of the sediments depending on the nature of the composition.

| | | Dry solids content of the |
|---|---|---|
| | Sediment volume after 1 | sediment after 24 Hour |
| composition | Hour dispersion (ml) | sedimentation (%) |
| 100% Riboflavin | 0.6 | 39.3% |
| (control) | | |
| Water-dispersible | 0.1 | 19.3% |
| pulverulent | | |
| composition A | | |

The sedimentation occurs more slowly with the pulverulent composition A of the invention compared to the control. The hydration of the sediment shows that the pulverulent composition A of the invention is more hydrated than the control and consequently better dispersed in the aqueous solution.

### Example 4

### Detailed Protocol for Manufacture of Water-Dispersible Pulverulent Composition B Obtained by Grinding Followed by Spray Drying

The ingredients of the composition are those disclosed in example 1.

0.76 kg of sodium octenyl succinate (OSA) and 2.26 kg of glucose syrup are added to 4.82 kg of water and complete dissolution is achieved. 2.16 kg of riboflavin are added and stirred vigorously to obtain a uniform suspension. The suspension is then grinded in a ball mixer (Wab laboratory device—Ball size 0.7-0.9 mm). The above suspension is then spray dried. The inlet air temperature is held at about 150° C. and the outlet temperature is about 100° C. The pulverulent composition B obtained disperses instantaneously in water and results in a stable suspension before uses.

### Example 5

### Stability of the Pulverulent Compositions A and B Obtained in Example 2 and 4

The stability is measured by the sedimentation test as follow:
10 ml of solution is prepared using 8.5 ml of demineralised water and 1.5 g of the pulverulent composition A or B in special sedimentation tubes graduated from 0.1 ml up to 10 ml;
complete dispersion is assumed by a 2 min vortex mixing;
sedimentation is controlled at defined times to evaluate the amount of sediment;
quality of the dispersion is evaluated by measuring:
   the dispersion of the solids all over the aqueous solution by measuring the height of the sediments of the hydrated particles. For the same amount of pulverulent composition, the higher the sediments, the more dispersed the particles in the aqueous solution
   the dry content of the sediment after 24 hours.

Results are described in table 4:

TABLE 4
Size of the sediment and percentage of dry content of the
sediment according to the nature of the composition.

| | | Sediment after | Dry content of the |
|---|---|---|---|
| Composition | | 24 H (ml) | sediment (%) |
| Riboflavine: Raw | | 1.5 | 39.4 |
| material (control) | | | |
| Composition of the | spray dried | 2.4 | 28.3 |
| invention A | | | |
| Composition of the | grinded and | 7.0 | 6.6 |
| invention B | spray dried | | |

Results show that the sediment of the composition A is more hydrated than the raw material (28% dry content compared to approx 39% dry content for the raw material). The comparison of the results for pulverulent composition A and B also demonstrates the effect of pre-grinding of the material by the important increase in moisture (only 6.6% solids) and size of the sediment (7 ml), demonstrating that the invention allows a more stable dispersion of the composition dispersed all over the aqueous dispersion.

### Example 6

### Ingredients of the Pulverulent Compositions C and D

The riboflavin water-dispersible pulverulent compositions C and D made in accordance with the process of the invention contain the ingredients disclosed in Table 5.

TABLE 5
Ingredients of the pulverulent compositions C and D.

| | % | |
|---|---|---|
| Ingredient | by total weight | Raw material reference |
| Riboflavin | 43.00 | Riboflavin (Hubei Guangji Pharmaceutical) |
| OSA | 57.00 | Sodium Octenyl Succinate starch (Cerestar) |

As a comparison, the pulverulent compositions C and D do not contain glucose syrup, unlike pulverulent compositions A and B as disclosed in the previous examples.

Pulverulent composition C was obtained after spray drying and pulverulent composition D was obtained after microgrinding followed by spray drying.

### Example 7

### Detailed Protocol for Manufacture of Water-Dispersible Pulverulent Compositions C and D

The ingredients of pulverulent compositions C and D are those disclosed in example 6.

The sodium octenyl succinate (OSA) is added to water and complete dissolution is achieved. Riboflavin is then added and stirred vigorously to obtain a uniform suspension.

One part of the preparation is then grinded in a ball mixer.

The above suspensions are then spray dried. The inlet air temperature is held at about 150° C. and the outlet temperature is about 100° C. The pulverulent compositions obtained disperse instantaneously in water and result in a stable suspension before uses.

Pulverulent composition C is the one after spray drying (without grinding).

Pulverulent composition D is the one after grinding and spray drying.

### Example 8

### Stability of the Pulverulent Compositions C and D Obtained in Example 7

Results of the sedimentation tests are disclosed in table 6.

TABLE 6
Size of the sediment and percentage of dry content of the sediment
according to the nature of the composition.

| | | Sediment | |
|---|---|---|---|
| | | after 24 H | Dry content of the |
| composition | Process | (ml) | sediment (%) |
| Riboflavin (raw | | 1.5 | 39.4 |
| material) (control) | | | |
| Pulverulent | Spray dried | 2.2 | 25.4 |
| Composition C | | | |
| Pulverulent | Grinded and Spray | 6.0 | 15.5 |
| Composition D | dried | | |

The results show that the sediment of the composition C is more hydrated than the raw material (25% dry content compared to approx. 39% dry content for the raw material). The comparison of the results for pulverulent compositions C and D also demonstrates the effect of pre-grinding of the material by the important increase in moisture (only 15.5% solids) and size of the sediment (6 ml). The composition using one saccharide allows good results for dispersion of the pulverulent composition in aqueous solution, but results are less effective than the composition A using a first saccharide and a second saccharide.

### Example 9

### Ingredients of Pulverulent Composition E

The riboflavin water-dispersible pulverulent composition E made in accordance with the invention contains the ingredients disclosed in table 7:

TABLE 7
Ingredients of pulverulent composition E.
Pulverulent composition E was obtained after spray drying.

| | % by | |
|---|---|---|
| Ingredient | total weight | Raw material reference |
| Riboflavin | 43.00 | Riboflavin (Hubei Guangji Pharmaceutical) |
| Maltodextrin | 57.00 | Maltodextrin (Roquette) |

### Example 10

### Detailed Protocol for Manufacture of Water-Dispersible Pulverulent Composition E

The maltodextrin is added to water and complete dissolution is achieved. Riboflavin is then added and stirred vigorously to obtain a uniform suspension. The above suspension is then spray dried. The inlet air temperature is held at about 150° C. and the outlet temperature is about 100° C. The pulverulent composition E obtained disperses instantaneously in water and results in a stable suspension before uses.

### Example 11

### Stability of the Pulverulent Composition E Obtained in Example 10

The results of the sedimentation tests are given in table 8.

TABLE 8
Size of the sediment and percentage of dry content of the sediment
according to the nature of the composition.

| | | Sediment after 24 H | Dry content of the |
|---|---|---|---|
| Composition | Process | (ml) | sediment (%) |
| Riboflavin (raw | | 1.5 | 39.4 |
| material) (control) | | | |
| Pulverulent | Spray dried | 1.3 | 32.6 |
| composition E | | | |

The results show that the sediments of pulverulent composition E is more hydrated than the raw material. Nevertheless results show that composition A using two saccharides gives better results in comparison to composition E using only one saccharide.

### Example 12

### Ingredients of Pulverulent Composition F

A riboflavin water-dispersible pulverulent composition F, made in accordance with the invention, contains the ingredients disclosed in table 9.

TABLE 9
Ingredients of pulverulent composition F.

| | % by | |
|---|---|---|
| Ingredient | total weight | Raw material reference |
| Riboflavin | 43.00 | Riboflavin (Hubei Guangji Pharmaceutical) |
| OSA | 14.25 | Sodium Octenyl Succinate starch (Cerestar) |
| Saccharose | 42.75 | Sugar (Beghin Say) |

As a comparison, the ingredients of pulverulent composition F are the same as those of the pulverulent composition A, except the nature of the second saccharide: glucose syrup for pulverulent composition A and saccharose for pulverulent composition F.

Pulverulent composition F was obtained after spray drying.

### Example 13

### Detailed Protocol for Manufacture of Water-Dispersible Pulverulent Composition F

The saccharose is added to water and complete dissolution is achieved. Riboflavin is then added and stirred vigorously to obtain a uniform suspension. The above suspension is then spray dried. The inlet air temperature is held at about 150° C. and the outlet temperature is about 100° C. The pulverulent composition F obtained disperses instantaneously in water and results in a stable suspension before uses.

### Example 14

### Stability of the Pulverulent Composition F Obtained in Example 13

The results of the sedimentation tests are given in table 10.

TABLE 10
Size of the sediment and percentage of dry content of the sediment
according to the nature of the composition.

| | | Sediment after 24 H | Dry content of the |
|---|---|---|---|
| Product | Process | (ml) | sediment (%) |
| Riboflavin (raw | | 1.5 | 39.4 |
| material) (control) | | | |
| Pulverulent | Spray dried | 1.6 | 34.0 |
| composition F | | | |

The tests show that the sedimentation is comparable between the pulverulent composition F and the control. The stability of the aqueous solution of pulverulent composition F is about the same as the one of pure riboflavin.

These results show that composition A using a second saccharides chosen among the glucose syrup types gives better results in comparison to composition F.

### Example 15

### Ingredients of Pulverulent Composition G

The riboflavin water-dispersible pulverulent composition made in accordance with the invention contains the ingredients disclosed in table 11.

TABLE 11
Ingredients of pulverulent composition G.

| | % by total | |
|---|---|---|
| Ingredient | weight | Raw material reference |
| Riboflavin | 43.00 | Riboflavin (Hubei Guangji |
| | | Pharmaceutical) |
| OSA | 14.25 | Sodium Octenyl Succinate starch |
| | | (Cerestar) |
| Fructooligosaccharide | 42.75 | fructooligosaccharide (Orafti) |

As a comparison, the ingredients of pulverulent composition G are the same than the one of pulverulent composition A and F, except the nature of the second saccharide: glucose syrup for pulverulent composition A, saccharose for pulverulent composition F and fructooligosaccharide for pulverulent composition G.

The pulverulent composition G was obtained after spray drying.

### Example 16

### Detailed Protocol for Manufacture of Water-Dispersible Pulverulent Composition G

The fructooligosaccharide is added to water and complete dissolution is achieved. Riboflavin is then added and stirred vigorously to obtain a uniform suspension. The above suspension is then spray dried. The inlet air temperature is held at about 150° C. and the outlet temperature is about 100° C. The pulverulent composition G obtained disperses instantaneously in water and results in a stable suspension before uses.

### Example 17

### Stability of the Pulverulent Composition G Obtained in Example 16

The results of sedimentation tests are given in table 12.

TABLE 12
Size of the sediment and percentage of dry content of the sediment
according to the nature of the composition.

| | | Sediment after 24 H | Dry content of the |
|---|---|---|---|
| Product | Process | (ml) | sediment (%) |
| Riboflavin (raw | | 1.5 | 39.4 |
| material) (control) | | | |
| Pulverulent | Spray dried | 2.1 | 31.1 |
| composition G | | | |

The results show that the sediment of the composition G is more hydrated than the raw material (31% dry content compared to approx 39% dry content for the raw material).

### Example 18

### Ingredients of Pulverulent Compositions H and I

The riboflavin water-dispersible pulverulent compositions H and I, made in accordance with the invention, contain the ingredients disclosed respectively in table 13 and 14.

TABLE 13
Ingredients of pulverulent composition H.

| | % by | |
|---|---|---|
| Ingredient | total weight | Raw material reference |
| Riboflavin | 95.00 | Riboflavin (Hubei Guangji Pharmaceutical) |
| CMC | 5.00 | Carboxymethylcellulose (Aqualon) |

TABLE 14
Ingredients of pulverulent composition I.

| | % by | |
|---|---|---|
| Ingredient | total weight | Raw material reference |
| Riboflavin | 70.00 | Riboflavin (Hubei Guangji Pharmaceutical) |
| CMC | 30.00 | Carboxymethylcellulose (Aqualon) |

The pulverulent compositions H and I were obtained after spray drying.

### Example 19

### Detailed Protocol for Manufacture of Water-Dispersible Pulverulent Compositions H and I

The CMC is added to water and complete dissolution is achieved. Riboflavin is then added and stirred vigorously to obtain a uniform suspension. The above suspensions are then spray dried. The inlet air temperature is held at about 150° C. and the outlet temperature is about 100° C. The pulverulent compositions H and I obtained disperse instantaneously in water and result in a stable suspension before uses.

### Example 20

### Stability of the Pulverulent Compositions H and I Obtained in Example 19

The results of sedimentation tests are given in table 15.

TABLE 15
Size of the sediment and percentage of dry content of the sediment
according to the nature of the composition.

| | | Sediment after 24 H | Dry content of the |
|---|---|---|---|
| Product | Process | (ml) | sediment (%) |
| Riboflavin (raw | | 1.5 | 39.4 |
| material) (control) | | | |
| Pulverulent | Spray dried | 3.1 | 32.2 |
| composition H | | | |
| Pulverulent | Spray dried | 5.4 | 13.3 |
| composition I | | | |

The results show that the sediments of the composition H and I are more hydrated than the raw material (respectively 32% and 13% dry content compared to approx 39% dry content for the raw material) and that hydration of the sediments is improved for pulverulent composition I compared to pulverulent composition H.

### Example 21

### Ingredients of Pulverulent Composition J

The riboflavin water-dispersible pulverulent composition J, made in accordance with the invention, contains the ingredients disclosed in table 16.

TABLE 16
Ingredients of pulverulent composition J.

| | % by total | |
|---|---|---|
| Ingredient | weight | Raw material reference |
| Riboflavin | 43.00 | Riboflavin (Hubei Guangji Pharmaceutical) |
| Arabic Gum | 57.00 | Acacia Gum (Colloïdes Naturals International) |

As a comparison, the pulverulent composition J contains Riboflavin and only one saccharide, the Arabic Gum, possessing hydrocolloidal properties.

The pulverulent composition J was obtained after spray drying.

### Example 22

### Detailed Protocol for Manufacture of Water-Dispersible Pulverulent Composition J Obtained in Example 21

The Arabic Gum is added to water and complete dissolution is achieved. Riboflavin is then added and stirred vigorously to obtain a uniform suspension. The above suspension is then spray dried. The inlet air temperature is held at about 150° C. and the outlet temperature is about 100° C. The pulverulent composition J obtained disperses instantaneously in water and results in a stable suspension before uses.

### Example 23

### Stability of the Pulverulent Composition J Obtained in Example 21

The results of sedimentation tests are given in table 17.

TABLE 17
Size of the sediment and percentage of dry content of the sediment
according to the nature of the composition.

| | | Sediment after 24 H | Dry content of the |
|---|---|---|---|
| Product | Process | (ml) | sediment (%) |
| Riboflavin (raw | | 1.5 | 39.4 |
| material) | | | |
| Pulverulent | Spray dried | 2.0 | 33.8 |
| composition J | | | |

The results show that the sediment of the composition J is more hydrated than the raw material (33% dry content compared to approx 39% dry content for the raw material). Nevertheless results show that composition A using two saccharides gives better results in comparison to composition E using only one saccharide.

### Example 24

### Ingredients of Pulverulent Composition K

The riboflavin water-dispersible pulverulent composition K, made in accordance with the invention, contains the ingredients disclosed in table 18.

TABLE 18
Ingredients of pulverulent composition K.

| | % by total | |
|---|---|---|
| Ingredient | weight | Raw material reference |
| Riboflavin | 95.00 | Riboflavin (Hubei Guangji Pharmaceutical) |
| Xanthan Gum | 5.00 | Xanthan Gum (Danisco) |

As a comparison, the pulverulent composition K contains Riboflavin and only one saccharide, the Xanthan Gum, possessing hydrocolloidal properties.

The pulverulent composition K was obtained after spray drying.

### Example 25

### Detailed Protocol for Manufacture of Water-Dispersible Pulverulent Composition J Obtained in Example 21

The Xanthan Gum is added to water and complete dissolution is achieved. Riboflavin is then added and stirred vigorously to obtain a uniform suspension. The above suspension is then spray dried. The inlet air temperature is held at about 150° C. and the outlet temperature is about 100° C. The pulverulent composition K obtained disperses instantaneously in water and results in a stable suspension before uses.

### Example 26

### Stability of the Pulverulent Composition K Obtained in Example 24

The results of sedimentation tests are given in table 19.

TABLE 19
Size of the sediment and percentage of dry content of the sediment
according to the nature of the composition.

| | | Sediment after 24 H | Dry content of the |
|---|---|---|---|
| Product | Process | (ml) | sediment (%) |
| Riboflavin (raw | | 1.5 | 39.4 |
| material) | | | |
| Pulverulent | Spray dried | 10.0 | 11.3 |
| composition K | | | |

The results show that the sediment of the composition K is more hydrated than the raw material (11% dry content compared to approx 39% dry content for the raw material).

### Example 27

### Ingredients of Composition L and M

The riboflavin water-dispersible pulverulent compositions L and M made in accordance with the invention contain the ingredients disclosed in table 20.

TABLE 20
Ingredients of pulverulent composition L and M.

| | % by total | |
|---|---|---|
| Ingredient | weight | Raw material reference |
| Riboflavin | 70.0 | Riboflavin (Hubei Guangji Pharmaceutical) |
| OSA | 7.5 | Sodium Octenyl Succinate starch (Cerestar) |
| Glucose syrup | 22.5 | Glucose sirup DE 38 (Cerestar) |

As a comparison, the ingredients of pulverulent composition L and M are the same as the one of pulverulent composition A and B, but in different concentrations (respectively 70% for L and M, and 43% of riboflavin for A and B).

The pulverulent composition L was obtained after spray drying and pulverulent composition M was obtained after microgrinding followed by spray drying.

### Example 28

### Detailed Protocol for Manufacture of Water-Dispersible Pulverulent Compositions L and M

The OSA and glucose syrup are added to water and complete dissolution is achieved. Riboflavin is then added and stirred vigorously to obtain a uniform suspension. One part of the preparation is then grinded in a ball mixer. The above suspensions are then spray dried. The inlet air temperature is held at about 150° C. and the outlet temperature is about 100° C. The pulverulent compositions obtained disperse instantaneously in water and result in a stable suspension before uses.

Pulverulent composition L is the one after spray drying (without grinding).

Pulverulent composition M is the one after grinding and spray drying.

### Example 29

### Stability of the Pulverulent Composition L Obtained in Example 28

Results of the sedimentation tests are given in table 21.

TABLE 21
Size of the sediment and percentage of dry content of the sediment
according to the nature of the composition.

| | | Sediment after 24 H | Dry content of the |
|---|---|---|---|
| Product | Process | (ml) | sediment (%) |
| Riboflavin (raw | | 1.5 | 39.4 |
| material) | | | |
| Pulverulent | Spray dried | 2.4 | 24.9 |
| composition L | | | |
| Pulverulent | Grinded and | 7.0 | 6.6 |
| composition M | Spray dried | | |

Results show that the sediment of the pulverulent composition L is more hydrated than the raw material (25% dry content compared to 39% dry content for the raw material)). The effect of pre-grinding of the material is also demonstrated by the important increase in moisture (only 6.6% solids) and size of the sediment (7 ml) demonstrating that the invention allows a more stable dispersion of the composition in aqueous dispersion.

1. Pulverulent composition comprising more than 15% in weight, and less than 96% in weight, preferably from 15 to 80%, and more preferably from 20% to 70%, of at least a vitamin having a solubility lower than 20 g/L and preferably 1 g/L and more preferably lower than 500 mg/L in aqueous media,
said pulverulent composition consisting in particles individually coated by at least one hydrophilic agent, and
said pulverulent composition being stable on storage and having instantaneous dispersion and solubility properties in aqueous solutions.

2. Pulverulent composition according to claim 1, wherein the vitamin is chosen among the group consisting of vitamin E, vitamin B2 (Riboflavin), vitamin D (D2 Ergocalciferol, D3 cholecalciferol), vitamin A and pro vitamin A, vitamin B8 (Biotin), vitamin B9 (Folic Acid), and vitamin B12 (cyanocobalamin and other cobalamin), preferably wherein the vitamin is riboflavin.

3. Pulverulent composition according to claim 1, wherein the hydrophilic agent comprises or is constituted by at least one saccharide, in particular chosen among the group consisting of monosaccharides, disaccharides and polysaccharides.

4. Pulverulent composition according to claim 3, wherein the hydrophilic agent comprises or consists in:
at least a first saccharide possessing hydrocolloidal properties,
said saccharide being preferably chosen among the group consisting of cellulose, alginate, starch, modified starch, in particular octenyl succinate starch, arabic gum, and,
at least a second saccharide, chosen among the group consisting of
monosaccharide such as fructose, glucose, galactose,
disaccharide such as saccharose, sucrose, lactose, maltose, trehalose and cellobiose, and,
polysaccharide such as glucose syrup, dextrin, inulin, cellulose, glycogen, starch and modified starch, fructooligosaccharide (FOS).

5. Pulverulent composition according to claim 1, wherein the pulverulent composition comprises from 5% to 85%, preferably 20% to 70%, and more preferably from 40% to 60% in weight of saccharide.

6. Pulverulent composition according to claim 4, wherein the vitamin is riboflavin, the first saccharide is octenyl succinate starch and the second saccharide is glucose syrup.

7. Pulverulent composition according to claim 1, characterized in that the pulverulent composition comprises granules, said granules consisting in a set of associated particles, and wherein
said granules comprise at least a vitamin having a solubility lower than 20 g/L and preferably 1 g/L and more preferably lower than 500 mg/L in aqueous media and at least a saccharide,
the mean diameter of said granule is from 10 μm to 500 μm, preferably 20 to 100 μm, more preferably 30 to 50 μm,
the span (DV05) of said granules is from 0.2 to 3.0, preferably 1.0 to 2.0, more preferably from 1.2 to 1.7,
the macroscopic density of said pulverulent composition is from 300 to 800 g/l, preferably 400 to 700 g/l, and more preferably 400 to 600 g/l,
the flow index determined by Flowdex method of said pulverulent composition is from 4 to 20, preferably from 5 to 10, more preferably around 8.

8. Pulverulent composition according to claim 7, wherein said granules comprise riboflavin, modified starch, in particular octenyl succinate starch and glucose syrup.

9. Pulverulent composition according to claim 1, characterized in that the pulverulent composition comprises granules, wherein:
said granules comprise at least a vitamin having a solubility lower than 20 g/L and preferably 1 g/L and more preferably lower than 500 mg/L in aqueous media and at least a saccharide,
the mean diameter of said granule is from 10 μm to 500 μm, preferably 20 to 100 μm, more preferably 30 to 50 μm,
the span (DV05) of said granules is from 0.2 to 3.0, preferably 1.0 to 2.0, more preferably from 1.2 to 1.7,
the macroscopic density of said pulverulent composition is from 300 to 800 g/l, preferably 400 to 700 g/l, and more preferably 400 to 600 g/l,
the flow index determined by Flowdex method of said pulverulent composition is from 4 to 20, preferably from 5 to 10, more preferably around 8,
the stability of a solution made of 1.5 g of said pulverulent composition in 8.5 g of water is such that a sediment from 1.5 ml to 10 ml, preferably 4 to 9 ml, more preferably from 6 to 8 ml is obtained after 24 hours.

10. Pulverulent composition according to claim 9, wherein said granules comprise riboflavin, modified starch, in particular octenyl succinate starch and glucose.

11. Granule comprising at least a vitamin having a solubility lower than 20 g/L and preferably 1 g/L and more preferably lower than 500 mg/L in aqueous media, and at least a saccharide wherein:
the mean diameter of said granule is from 10 μm to 500 μm, preferably 20 to 100 μm, more preferably 30 to 50 μm,
the macroscopic density of said granule is from 300 to 800 g/l, preferably 400 to 700 g/l, and more preferably 400 to 600 g/l,
the time to disperse and solubilize 10 grams of said granules in 100 mL of an aqueous solutions is from 2 min to 5 s, preferably 1 min to 5 s, and more preferably less than 1 min. said granule comprising preferably riboflavin, glucose and modified starch, in particular octenyl succinate starch.

12. Process of preparation of pulverulent compositions according to claim 1 comprising a step of atomisation of an aqueous phase comprising a vitamin and at least one saccharide, said vitamin having a solubility lower than 20 g/L and preferably 1 g/L and more preferably lower than 500 mg/L in aqueous media.

13. Process of preparation of pulverulent compositions according to claim 1 comprising:
a step of mixing a vitamin having a solubility lower than 20 g/L and preferably 1 g/L and more preferably lower than 500 mg/L in an aqueous media with at least a saccharide to obtain an aqueous phase comprising a vitamin and at least one saccharide, and,
a step of atomisation of said aqueous phase comprising a vitamin and at least one saccharide to obtain a mixture comprising a vitamin and at least one saccharide, said mixture being constituted by granules, and,
possibly a step of coating of said granules to obtain a pulverulent composition.

14. Process according to claim 12 in which a step of microgrinding of the aqueous phase comprising a vitamin and at least one saccharide is added prior to the step of atomisation.

15. Process of preparation of pulverulent compositions according to claim 1 comprising:
a step of mixing a vitamin having a solubility lower than 20 g/L and preferably 1 g/L and more preferably lower than 500 mg/L in aqueous media with at least a saccharide to obtain an aqueous phase comprising a vitamin and at least one saccharide, and,
possibly a step of microgrinding of the aqueous phase comprising a vitamin and at least one saccharide to obtain a microgrinded aqueous phase comprising a vitamin and at least one saccharide, and,
a step of atomisation of the microgrinded aqueous phase comprising a vitamin and at least one saccharide to obtain a mixture comprising a vitamin and at least one saccharide, said mixture being constituted by granules, and,
possibly a step of coating of said granules to obtain a pulverulent composition, and,
a step of recovery of the pulverulent composition obtained.

16. (canceled)

17. Liquid food product containing:
from 0.1 mg/kg to 1500 mg/kg, preferably 1 mg/kg to 400 mg/kg, more preferably 4 mg/kg to 40 mg/kg of a pulverulent composition according to claim 1,
from 0.1 mg/kg to 1500 mg/kg, preferably 1 mg/kg to 400 mg/kg, more preferably 4 mg/kg to 40 mg/kg of granules comprising at least a vitamin having a solubility lower than 20 g/L in aqueous media, and at least a saccharide wherein:
the mean diameter of said granule is from 10 μm to 500 μm,
the macroscopic density of said granule is from 300 to 800 g/l,
the time to disperse and solubilize 10 grams of said granules in 100 mL of an aqueous solutions is from 2 min to 5 s,
said granule comprising riboflavin, glucose and modified starch,
wherein:
said liquid food product is liquid or frozen,
said liquid food product is stable at temperatures from 6 to 60° C., preferably from 10° C. to 50° C., more preferably from 20° C. to 40° C. during a period of 2 years,
said pulverulent composition or granule is homogeneously dispersed in said liquid food product,
said vitamin does not form any deposit on the wall of a container which would contain said liquid food product,
said liquid food product being preferably chosen among the group consisting of oil, infantile milk, milk, acidified milk products, drinks, fruit juices and sorbet,
except liquid food product chosen from the group consisting of infantile milk, milk, acidified milk products, drinks and oils and containing:
either a pulverulent composition,
or granules,
said pulverulent composition or said granules comprising:
riboflavin,
a first saccharide possessing hydrocolloidal properties, and/or
a second saccharide, chosen among the group consisting of
monosaccharide such as fructose, glucose, galactose,
disaccharide such as saccharose, sucrose, lactose, maltose, trehalose and cellobiose, and,
polysaccharide such as glucose syrup, dextrin, inulin, cellulose, glycogen, starch and modified starch, fructooligosaccharide (FOS).

18. Solid food product containing:
from 1 mg/kg to 1000 g/kg, preferably 10 mg/kg to 500 g/kg, more preferably 0.1 g/kg to 100 g/kg of a pulverulent composition according to claim 1, or,
from 1 mg/kg to 1000 g/kg, preferably 10 mg/kg to 500 g/kg, more preferably 0.1 g/kg to 100 g/kg of granules comprising at least a vitamin having a solubility lower than 20 g/L in aqueous media, and at least a saccharide wherein:
the mean diameter of said granule is from 10 μm to 500 μm,
the macroscopic density of said granule is from 300 to 800 g/l,
the time to disperse and solubilize 10 grams of said granules in 100 mL of an aqueous solutions is from 2 min to 5 s,
said granule comprising riboflavin, glucose and modified starch,
wherein:
said solid food product is stable at temperatures from 6 to 60° C., preferably from 10° C. to 50° C., more preferably from 20° C. to 40° C. during a period of 2 years,
said pulverulent composition or granule is homogeneously dispersed in said solid food product,
said solid food product being preferably chosen among the group consisting of confectionery, bakery, noodles, bread, rice, cereals,
except solid food product containing:
either a pulverulent composition,
or granules,
said pulverulent composition or said granules comprising:
riboflavin,
a first saccharide possessing hydrocolloidal properties, and/or
a second saccharide, chosen among the group consisting of
monosaccharide such as fructose, glucose, galactose,
disaccharide such as saccharose, sucrose, lactose, maltose, trehalose and cellobiose, and,
polysaccharide such as glucose syrup, dextrin, inulin, cellulose, glycogen, starch and modified starch, fructooligosaccharide (FOS).

19. Semi-liquid food product containing:
from 0.1 mg/kg to 1500 mg/kg, preferably 1 mg/kg to 400 mg/kg, more preferably 4 mg/kg to 40 mg/kg of a pulverulent composition according to claim 1,
from 0.1 mg/kg to 1500 mg/kg, preferably 1 mg/kg to 400 mg/kg, more preferably 4 mg/kg to 40 mg/kg of granules comprising at least a vitamin having a solubility lower than 20 g/L in aqueous media, and at least a saccharide wherein:
the mean diameter of said granule is from 10 μm to 500 μm,
the macroscopic density of said granule is from 300 to 800 g/l,
the time to disperse and solubilize 10 grams of said granules in 100 mL of an aqueous solutions is from 2 min to 5 s,
said granule comprising riboflavin, glucose and modified starch,
wherein:
said semi-liquid food product is semi-liquid or frozen,
said semi-liquid food product is stable at temperatures from 6 to 60° C., preferably from 10° C. to 50° C., more preferably from 20° C. to 40° C. during a period of 2 years,
said pulverulent composition or granule is homogeneously dispersed in said semi-liquid food product,
said semi-liquid food product being preferably chosen among the group consisting of spreads, jams, yoghourts, dips and ice creams,
except semi-liquid food product chosen from the group consisting of spreads and jams and containing:
either a pulverulent composition,
or granules,
said pulverulent composition or said granules comprising:
riboflavin,
a first saccharide possessing hydrocolloidal properties, and/or
a second saccharide, chosen among the group consisting of
monosaccharide such as fructose, glucose, galactose,
disaccharide such as saccharose, sucrose, lactose, maltose, trehalose and cellobiose, and,
polysaccharide such as glucose syrup, dextrin, inulin, cellulose, glycogen, starch and modified starch, fructooligosaccharide (FOS).

20. Emulsion food product containing:
from 0.1 mg/kg to 1500 mg/kg, preferably 1 mg/kg to 400 mg/kg, more preferably 4 mg/kg to 40 mg/kg of a pulverulent composition according to claim 1,
from 0.1 mg/kg to 1500 mg/kg, preferably 1 mg/kg to 400 mg/kg, more preferably 4 mg/kg to 40 mg/kg of granules comprising at least a vitamin having a solubility lower than 20 g/L in aqueous media, and at least a saccharide wherein:
the mean diameter of said granule is from 10 μm to 500 μm,
the macroscopic density of said granule is from 300 to 800 g/l,
the time to disperse and solubilize 10 grams of said granules in 100 mL of an aqueous solutions is from 2 min to 5 s,
said granule comprising riboflavin, glucose and modified starch,
wherein:
said emulsion food product is liquid or frozen,
said emulsion food product is stable at temperatures from 6 to 60° C., preferably from 10° C. to 50° C., more preferably from 20° C. to 40° C. during a period of 2 years,
said pulverulent composition or granule is homogeneously dispersed in said emulsion food product,
said emulsion food product is a sauce, in particular mayonnaise and mustard.

21. Powder containing:
from 1 mg/kg to 999 g/kg, preferably 10 mg/kg to 500 g/kg, more preferably 0.1 g/kg to 100 g/kg of a pulverulent composition according to claim 1, or,
from 1 mg/kg to 999 g/kg, preferably 10 mg/kg to 500 mg/kg, more preferably 0.1 g/kg to 100 g/kg of granules comprising at least a vitamin having a solubility lower than 20 g/L in aqueous media, and at least a saccharide wherein:
the mean diameter of said granule is from 10 μm to 500 μm,
the macroscopic density of said granule is from 300 to 800 g/l,
the time to disperse and solubilize 10 grams of said granules in 100 mL of an aqueous solutions is from 2 min to 5 s,
said granule comprising riboflavin, glucose and modified starch, and,
at least a foodstuff being preferably chosen among the group consisting of dehydrated soup, dehydrated sauce, dehydrated drink preparation, powder milk, cocoa powder, instant powder drinks, meal replacement products and effervescent products,
wherein:
said powder is stable at temperatures from 6 to 60° C., preferably from 10° C. to 50° C., more preferably from 20° C. to 40° C. during a period of 2 years,
said pulverulent composition or granule is homogeneously dispersed in said powder,
said powder is liable to be instantaneously dispersed in an aqueous solution to provide a beverage,
except powders containing:
either a pulverulent composition or granules, said pulverulent composition or said granules comprising:
riboflavin,
a first saccharide possessing hydrocolloidal properties, and/or
a second saccharide, chosen among the group consisting of
monosaccharide such as fructose, glucose, galactose,
disaccharide such as saccharose, sucrose, lactose, maltose, trehalose and cellobiose, and,
polysaccharide such as glucose syrup, dextrin, inulin, cellulose, glycogen, starch and modified starch, fructooligosaccharide (FOS), and,
a foodstuff being chosen among the group consisting of powder milk, cocoa powder, instant powder drinks, meal replacement products and effervescent products.

22. Powder according to claim 21, wherein said foodstuff is soluble in an aqueous solution and preferably chosen among the group consisting of dehydrated drink preparation, instant powder drinks and effervescent products, and wherein said powder is liable to be dissolved in an aqueous solution to provide a beverage.

23. Process of preparation of a liquid food product, comprising a step of instantaneously dispersing a powder according to claim 21 in an aqueous solution.

24. Process according to claim 23, comprising an additional phase of dissolving a powder in an aqueous solution.

25. Pharmaceutical composition containing:
an active substance contained in the pulverulent composition according to according to claim 1, or,
an active substance contained in granules comprising at least a vitamin having a solubility lower than 20 g/L in aqueous media, and at least a saccharide wherein:
the mean diameter of said granule is from 10 μm to 500 μm,
the macroscopic density of said granule is from 300 to 800 g/l,
the time to disperse and solubilize 10 grams of said granules in 100 mL of an aqueous solutions is from 2 min to 5 s,
said granule comprising riboflavin, glucose and modified starch,
in association with a pharmaceutically acceptable vehicule.

26. Pharmaceutical composition according to claim 25, wherein the pulverulent composition or the granules comprise(s) riboflavin.
